(19) United States
(12) Patent Application Publication
(10) Pub. No.: US 2006/0167147 A1
(43) Pub. Date: Jul. 27, 2006

(54) Metal-containing composite materials

(75) Inventor: Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(73) Assignee: Blue Membranes GmbH

(21) Appl. No.: 11/339,161

(22) Filed: Jan. 24, 2006

Related U.S. Application Data
(60) Provisional application No. 60/646,912, filed on Jan. 24, 2005.

(51) Int. Cl. C03C 3/00; C08L 1/10; C09D 11/02; B01J 19/06; A61K 9/50; A61K 9/14; C08K 5/54; H01B 3/44; C08L 1/12; C07F 7/12; B60C 1/00; C03C 25/24; C09D 183/04; A61K 9/70; C04B 41/50; C07F 7/18; C04B 41/49; A61K 9/127

(52) U.S. Cl. 524/174; 524/175; 524/251; 524/261; 524/263; 524/264; 524/265; 524/266; 524/267; 524/268; 524/269; 501/12; 106/170.13; 106/170.2; 106/171.1; 106/287.1; 106/287.13; 106/287.14; 106/287.15; 106/287.16; 424/450; 424/489; 424/490; 424/494; 424/497

(57) ABSTRACT

The present invention relates to a process for the manufacture of metal-containing materials or composite materials, the process comprising the steps of encapsulating at least one metal-based compound in a polymeric shell, thereby producing a polymer-encapsulated metal-based compound and/or coating a polymeric particle with at least one metal-based compound; forming a sol from suitable hydrolytic or non-hydrolytic sol/gel forming components; combining the polymer-encapsulated metal-based compound and/or the coated polymeric particle with the sol, thereby producing a combination thereof; and converting the combination into a solid metal-containing material. The present invention further relates to metal-containing materials produced in accordance with the above process.

### CROSS-REFERENCE TO RELATED APPLICATION(S)

This application claims priority from U.S. Patent Application No. 60/646,912, filed Jan. 24, 2005, the entire disclosure of which is incorporated herein by reference.

### BACKGROUND OF THE INVENTION

Porous metal-based ceramic materials like cermets are typically used as components for friction-type bearings, filters, fumigating devices, energy absorbers or flame barriers. Constructional elements having hollow space profiles and increased stiffness are important in construction technology. Porous metal-based materials are becoming increasingly important in the field of coatings, and the functionalization of such materials with specific physical, electrical, magnetic and optical properties is of major interest. Furthermore, these materials can play an important role in applications such as photovoltaics, sensor technology, catalysis, and electro-chromatic display techniques.

Generally, there may be a need for porous metal-based materials having nano-crystalline fine structures, which allow for an adjustment of the electrical resistance, thermal expansion, heat capacity and conductivity, as well as superelastic properties, hardness, and mechanical strength.

Furthermore, there may be a need for porous metal-based materials which may be produced in a cost efficient manner. Conventional porous metal-based materials and cermets can be produced by powder- or melt-sintering methods, or by infiltration methods. Such methods can be technically and economically complex and costly, particularly since the control of the desired material properties can often depend on the size of the metal particles used. This parameter may not always be adjustable over an adequate range in certain applications like coatings, where process technology such as powder coating or tape casting may be used. According to conventional methods, porous metals and metal-based materials may typically be made by the addition of additives or by foaming methods, which normally require a pre-compacting of the green body.

Also, there may be a need for porous metal-based materials, where the pore size, the pore distribution and the degree of porosity can be adjusted without deteriorating the physical and chemical properties of the material. Conventional methods based on fillers or blowing agents, for example, can provide porosity degrees of 20-50%. However, the mechanical properties such as hardness and strength may decrease rapidly with increasing degree of porosity. This may be particularly disadvantageous in biomedical applications such as implants, where anisotropic pore distribution, large pore sizes, and a high degree of porosity are required, together with long-term stability with respect to biomechanical stresses.

In the field of biomedical applications, it may be important to use biocompatible materials. For example, metal-based materials for use in drug delivery devices, which may be used for marking purposes or as absorbents for radiation, can preferably have a high degree of functionality and may combine significantly different properties in one material. In addition to specific magnetical, electrical, dielectrical or optical properties, the materials may have to provide a high degrees of porosity in suitable ranges of pore sizes.

The sol/gel-process technology can be widely applied to build up different types of material networks. The linkage of the components under formation of the sol or gel can take place in several ways, e.g., via conventional hydrolytic or non-hydrolytic sol/gel-processing. Certain exemplary embodiment of the present invention may utilize sol/gel technology to produce metal-containing composite materials.

A “sol” can be a dispersion of colloidal particles in a liquid, and the term “gel” may connote an interconnected, rigid network of pores of submicrometer dimensions and polymeric chains whose average length is typically greater than a micrometer. For example, the sol/gel-process may involve mixing of the precursors, e.g. sol/gel forming components, into a sol, adding further additives or materials, casting the mixture in a mold or applying the sol onto a substrate in the form of a coating, gelation of the mixture whereby the colloidal particles are linked together to become a porous three-dimensional network, aging of the gel to increase its strength; converting the gel into a solid material by drying from liquid and/or dehydration or chemical stabilisation of the pore network, and densification of the material to produce structures with ranges of physical properties. Such processes are described, for example, in Henge and West, The Sol/Gel-Process, 90 Chem. Ref. 33 (1990).

The term “sol/gel” as used within the specification may mean either a sol or a gel. The sol can be converted into a gel as mentioned above, e.g. by aging, curing, raising of pH, evaporation of solvent or by any other conventional methods.

A sol/gel-processing technology generally provides several possibilities for cost efficient low temperature production of biocompatible materials with a wide range of individually adjustable properties, allowing a tailoring of the properties of the individually produced material. For example, silica-xerogels, which are partially hydrolysed oxides of silicon, can be produced by sol/gel-processing techniques that have conventionally been used to produce ceramic and glassy materials. The sol/gel-process can be primarily based on a hydrolysation of a metal alkoxide and subsequent polymerisation/polycondensation of the metal hydroxides. When the polymerisation reaction proceeds, chains, rings, and three dimensional networks may be formed, and a gel, typically comprising water and the alcohol of the alkoxy groups of the alkoxides, is formed. The so-formed gel may then be converted by a drying or heating step into a solid material. Since there may be a large variety of possible additives to be added to sols in the sol/gel technology, such technology can provide a large variety of possibilities to modify the composition and the properties of the materials produced.

European Patent Publication EP 0 680 753 describes a sol/gel produced silica coating and particles containing a biologically active substance, where the release rate of an active agent incorporated therein can be controlled by addition of penetration agents such as polyethylene glycol and sorbitol.

U.S. Pat. No. 5,074,916 describes sol/gel-process techniques used for the production of alkali free bioactive glass compositions based on SiO₂, CaO and P₂O₅.

International Patent Publication WO 96/03117 describes bone bioactive controlled release carriers comprising silica-based glass providing for the controlled release of biologically active molecules, their methods of preparation and methods of use.

U.S. Pat. No. 6,764,690 describes controllably dissolvable silica-xerogels prepared by a sol/gel-process and their use for drug delivery devices comprising the controllably dissolvable silica-xerogels prepared by a sol/gel-process into which structure biologically active agents can be incorporated.

### SUMMARY OF EXEMPLARY EMBODIMENTS OF THE INVENTION

It is one object of the present invention to provide, e.g., a material based on metallic and ceramic precursors which can be modifiable in its properties and composition, which allows for the tailoring of the mechanical, thermal, electrical, magnetical and optical properties thereof. Another object of the present invention is to provide, e.g., metal-containing composite materials such that the porosity of the formed material can be varied for use in a large range of application fields without adversely affecting the physical and chemical stability.

A further object of the present invention is to provide, e.g., a new material and process for the production thereof, which may be used as a coating as well as a bulk material. Yet another object of the present invention is to provide, e.g., a method for the production of composite material in which the conversion of the sol/gel into the composite material allows a robust and relatively error-free sintering process to achieve extremely stable materials.

An exemplary embodiment of the present invention relates to a composition of matter and, for example, to metal-containing composite materials composed of organic and inorganic components. Another exemplary embodiment of the present invention is further directed to a process for the manufacture of metal-containing materials. Metal-based compounds can be encapsulated in a polymeric shell and the polymer-encapsulated metal-based compounds can be combined with a sol in a conventional sol/gel-process technology, and the combination can be subsequently converted into a solid metal-containing material.

Still another object of the present invention is to provide, e.g., a material obtainable by a process such as those described above, which may be in the form of a coating or in the form of a porous bulk material.

A still further object of the present invention is to provide a material containing metal, obtainable by the process as described above, which may have bioerodible properties, or may be at least partially dissolvable in the presence of physiologic fluids.

Yet a further object of the present invention is to provide, e.g., such metal-containing materials for use in the biomedical field, in the form of implants, drug delivery devices, or coatings for implants and drug delivery devices, and the like.

For example, these and other objects of the invention can be achieved by one exemplary embodiment of the present invention, which provides a process for the manufacture of metal-containing materials, such that the process comprises the following steps in no specific order:
a) encapsulating at least one metal-based compound in a polymeric shell, thereby producing a first composition comprising a polymer-encapsulated metal-based compound;
b) forming a sol from hydrolytic or non-hydrolytic sol/gel forming components;
c) combining the polymer-encapsulated metal-based compound and the sol to produce a second composition; and
d) converting the second composition into a solid metal-containing material.

In a further exemplary embodiment of the present invention, a process for the manufacture of metal containing materials or composite materials is provided, such that the process comprises the following steps in no specific order:
a) providing a first composition comprising a polymeric particle coated with at least one metal-based compound;
b) forming a sol from hydrolytic or non-hydrolytic sol/gel forming components;
c) combining the coated polymeric particle and the sol to produce a second composition; and
d) converting the second composition into a solid metal-containing material.

In further exemplary embodiments of the present invention, the metal-based compound used in processes such as those described above may be provided in the form of a colloidal particle, a nanocrystalline or microcrystalline particle, or a nanowire.

In yet another exemplary embodiment of the present invention, the metal-basaed compound may be encapsulated in several layers or shells of organic material, or in a vesicle, a liposome, a micelle, or an overcoat of a suitable material.

In yet another exemplary embodiment of the present invention, additives may be added to the first composition, the sol/gel forming components, and/or to the second composition used in processes such as those described above. These additives can be biologically or therapeutically active compounds, fillers, surfactants, pore-forming agents, plasticizers, lubricants, and the like.

In still another exemplary embodiment of the present invention, the second composition can be converted to a metal-containing composite material by drying, pyrolysis, sintering, or other heat treatments, and the conversion may be performed under reduced pressure or in a vacuum.

In another exemplary embodiment of the present invention, fillers may be added to the first composition, the sol/gel forming components, and/or to the second composition used in processes such as those described above. These fillers may then be removed completely or partially from the solid metal-containing material produced in processes such as those described above. Removal of the fiillers can be achieved by dissolving them or thermally decomposing them, either completely or partially.

Still further exemplary embodiments of the present invention provide metal-containing composite materials which may be produced using processes such as those described above. Such materials may be in the form of bulk compositions, or they may be provided as coatings on substrates or devices. These materials may further be bioerodible or at least partially dissolvable when exposed to physiologic fluids.

### DETAILED DESCRIPTION OF EXEMPLARY EMBODIMENTS OF THE INVENTION

Metal-containing materials according to ccertain exemplary embodiments of the present invention may exhibit advantageous properties, e.g., they can be processed from sols and/or gels with litrtle or no mass- and/or volume shrinkage at low temperatures. For example, sols and combinations prepared in accordance with certain exemplary embodiments of the present invention may be suitable for coating of almost any type of substrate with porous or non-porous film coatings, which may then be converted into metal-containing materials. Coatings as well as shaped bulk materials can be obtained by such processes.

Metal-Based Compounds

According to certain exemplary embodiments of the present invention, metal-based compounds may be initially encapsulated in a polymer material.

For example, the metal-based compounds may be selected from zero-valent metals, metal alloys, metal oxides, inorganic metal salts, particularly salts from alkaline and/or alkaline earth metals and/or transition metals, preferably alkaline or alkaline earth metal carbonates, sulphates, sulfites, nitrates, nitrites, phosphates, phosphites, halides, sulfides, oxides, as well as mixtures thereof; organic metal salts, particularly alkaline or alkaline earth and/or transition metal salts, in particular their formiates, acetates, propionates, malates, maleates, oxalates, tartrates, citrates, benzoates, salicylates, phtalates, stearates, phenolates, sulfonates, and amines as well as mixtures thereof; organometallic compounds, metal alkoxides, semiconductive metal compounds, metal carbides, metal nitrides, metal oxynitrides, metal carbonitrides, metal oxycarbides, metal oxynitrides, and metal oxycarbonitrides, preferably of transition metals; metal-based core-shell nanoparticles, preferably with CdSe or CdTe as the core and CdS or ZnS as the shell material; metal-containing endohedral fullerenes and/or endometallofullerenes, preferably of rare earth metals like cerium, neodymium, samarium, europium, gadolinium, terbium, dysprosium, holmium; as well as any combinations of any of the foregoing.

Also, biodegradable metal-based compounds selected from alkaline or alkaline earth metal salts or compounds can be used, such as magnesium-based or zinc-based compounds or the like or nano-alloys or any mixture thereof. The metal-based compound used in certain exemplary embodiments of the present invention mat be selected from magnesium salts, oxides or alloys, which can be used in biodegradable coatings or molded bodies, including in the form of an implant or a coating on an implant, that may be capable of degradation when exposed to bodily fluids, and which may further result in formation of magnesium ions and hydroxyl apatite.

In the exemplary embodiments of the present invention, the metal-based compounds of the above mentioned materials may be provided in the form of nano- or microcrystalline particles, powders or nanowires. The metal-based compounds may have an average particle size of about 0.5 nm to 1,000 nm, preferably about 0.5 nm to 900 nm, or more preferably from about 0.7 nm to 800 nm.

The metal-based compounds to be encapsulated can also be provided as mixtures of metal-based compounds, particularly nanoparticles thereof having different specifications, in accordance with the desired properties of the metal-containing material to be produced. The metal-based compounds may be used in the form of powders, in solutions, suspensions or dispersions in polar, non-polar or amphilic solvents, solvent mixtures or solvent-surfactant mixtures, or emulsions.

Nanoparticles of the above-mentioned metal-based compounds may be easier to modify due to their high surface-to-volume ratio. The metal-based compounds, particularly nanoparticles, may for example be modified with hydrophilic ligands, e.g., with trioctylphosphine, in a covalent or non-covalent manner.

Examples of ligands that may be covalently bonded to metal nanoparticles include fatty acids, thiol fatty acids, amino fatty acids, fatty acid alcohols, fatty acid ester groups of mixtures thereof, for example, oleic acid and oleylamine, and similar conventional organometallic ligands.

The metal-based compounds may be selected from metals or metal-containing compounds, for example hydrides, inorganic or organic salts, oxides and the like. Depending on the conversion conditions and the process conditions used in the exemplary embodiments of the present invention, oxidic as well as zero-valent metals may be produced from metal compounds used in the process. It has been found that alloys, ceramic materials and composite materials may be produced from metal-based compounds, particularly metal-based nanoparticles, wherein the porosity may be adjusted over wide ranges in accordance with further additives used, their structure, molecular weight and solids content, and the metal-based compound content. It has also been found that by combining polymer encapsulated metal-based compounds, particularly of nano-size, and sols conventionally used in sol/gel process technology, materials may be produced wherein one or more of the mechanical, tribological, electrical and/or optical properties may be adjusted by controlling these solids content and the composition of the metal-based nanoparticles. The resulting material propertiess may depend on the primary or average particle size and the structure of these encapsulated metal-based compounds.

Furthermore, the use of alkoxides in combination with polymer encapsulated metal-based compounds may lead to hybride ceramic composites. The thermal expansion coefficient of these composites may be adjusted by suitably selecting the metals or metal compounds used and their solids content in the sol/gel. Additionally, the selection of the alkoxides used in the sol and the proper selection of the atmosphere during the conversion steps, as described herein below, may lead to a reduction of the volume shrinkage and to the production of stable aerogels and xerogels.

Certain metal-based compounds may include, but are not limited to, powders, preferably nanomorphous nanoparticles, of zero-valent-metals, metal oxides or combinations thereof, e.g. metals and metal compounds selected from the main group of metals in the periodic table, transition metals such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals. The metal-based compounds which may be used include, e.g., iron, cobalt, nickel, manganese or mixtures thereof, such as iron-platinum-mixtures. Magnetic metal oxides may also be used, such as iron oxides and ferrites. To provide materials having magnetic or signaling properties, magnetic metals or alloys may be used, such as ferrites, e.g. gamma-iron oxide, magnetite or ferrites of Co, Ni, or Mn. Examples of such materials are described in International Patent Publications WO83/03920, WO83/01738, WO88/00060, WO85/02772, WO89/03675, WO90/01295 and WO90/01899, and U.S. Pat. Nos. 4,452,773, 4,675,173 and 4,770,183.

Additionally, semiconducting compounds and/or nanoparticles may be used in further exemplary embodiments of the present invention, including semiconductors of groups II-VI, groups III-V, or group IV of the periodic system. Suitable group I-VI-semiconductors include, for example, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe or mixtures thereof. Examples of group III-V semiconductors include, for example, GaAs, GaN, GaP, GaSb, InGaAs, InP, InN, InSb, InAs, AlAs, AlP, AlSb, AlS, or mixtures thereof. Examples of group IV semiconductors include germanium, lead and silicon. Also, combinations of any of the foregoing semiconductors may be used.

In certain exemplary embodiments of the present invention, it may be preferable to use complex metal-based nanoparticles as the metal-based compounds. These may include, for example, so-called core/shell configurations, which are described by Peng et al., Epitaxial Growth of Highly Luminescent CdSe/CdS Core/Shell Nanoparticles with Photostability and Electronic Accessibility, Journal of the American Chemical Society (1997, 119: 7019-7029).

Semiconducting nanoparticles may be selected from those materials listed above, and they may have a core with a diameter of about 1 to 30 nm, or preferably about 1 to 15 nm, upon which further semiconducting nanoparticles may be crystallized to a depth of about 1 to 50 monolayers, or preferably about 1 to 15 monolayers. Cores and shells may be present in combinations of the materials listed above, including CdSe or CdTe cores, and CdS or ZnS shells.

In a further exemplary embodiment of the present invention, the metal-based compounds may be selected based on their absorptive properties for radiation in a wavelength ranging anywhere from gamma radiation up to microwave radiation, or based on their abiltiy to emit radiation, particularly in the wavelength region of about 60 nm or less. By suitably selecting the metal-based compounds, materials having non-linear optical properties may be produced. These include, for example, materials that can block IR-radiation of specific wavelengths, which may be suitable for marking purposes or to form therapeutic radiation-absorbing implants. The metal-based compounds, their particle sizes and the diameter of their core and shell may be selected to provide photon emitting compounds, such that the emission is in the range of about 20 nm to 1000 nm. Alternatively, a mixture of suitable compounds may be selected which emits photons of differing wavelengths when exposed to radiation. In one exemplary embodiment of the present invention, fluorescent metal-based compounds may be selected that do not require quenching.

Metal-based compounds that may be used in further exemplary embodiments of the present invention include nanoparticles in the form of nanowires, which may comprise any metal, metal oxide, or mixtures thereof, and which may have diameters in the range of about 2 nm to 800 nm, or preferably about 5 nm to 600 nm.

In further exemplary embodiments of the present invention, the metal-based compound may be selected from metallofullerenes or endohedral carbon nanoparticles comprising almost any kind of metal compound such as those mentioned above. Particularly preferred are endohederal fullerenes or endometallofullerenes, respectively, which may comprise rare earth metals such as cerium, neodynium, samarium, europium, gadolinium, terbium, dysprosium, holmium and the like. Endohedralmetallofullerenes may also comprise transition metals as described above. Suitable endohedral fullerenes, e.g. those which may be used for marker purposes, are further described in U.S. Pat. No. 5,688,486 and International Patent Publication WO 93/15768. Carbon-coated metal nanoparticles comprising, for example, carbides may be used as the metal-based compound. Also, metal containing nanomorphous carbon species such as nanotubes, onions; as well as metal-containing soot, graphite, diamond particles, carbon black, carbon fibres and the like may also be used in other exemplary embodiments of the present invention.

The metal-based compounds as described above may be encapsulated in a polymeric shell. The encapsulation of the metal-based compounds into polymers may be achieved by various coventional polymerization technique, e.g. dispersion-, suspension- or emulsion-polymerization. Preferred encapsulating polymers include, but are not limited to, polymethylmethacrylate (PMMA), polystyrol or other latex-forming polymers, polyvinyl acetate, or conducting polymers. These polymer capsules, which contain the metal-based compounds, can further be modified, for example by linking lattices and/or further encapsulation with polymers, or they can be further coated with elastomers, metal oxides, metal salts or other suitable metal compounds, e.g. metal alkoxides. Conventional techniques may optionally be used to modify the polymers, and may be employed depending on the requirements of the individual compositions to be used. The use of encapsulated metal-based compounds may prevent or inhibit aggregation, so that the encapsulated precursor material can be processed in a sol/gel process without agglomerating and/or adversely affecting the resulting composite material.

The encapsulation of the metal-based compounds can lead to covalently or non-covalently encapsulated metal-based compounds, depending on the individual materials used. For combining with the sol, the encapsulated metal-based compounds may be provided in the form of polymer spheres, particularly microspheres, or in the form of dispersed, suspended or emulgated particles or capsules. Conventional methods suitable for providing or manufacturing encapsulated metal-based compounds, dispersions, suspensions or emulsions, particularly preferred mini-emulsions, thereof can be utilize. Suitable encapsulation methods are described, for example, in Australian publication AU 9169501, European Patent Publications EP 1205492, EP 1401878, EP 1352915 and EP 1240215, U.S. Pat. No. 6,380,281, U.S. Patent Publication 2004192838, Canadian Patent Publication CA 1336218, Chinese Patent Publication CN 1262692T, British Patent Publication GB 949722, and German Patent Publication DE 10037656; and in S. Kirsch, K. Landfester, O. Shaffer and M. S. El-Aasser, “Particle morphology of carboxylated poly-(n-butyl acrylate)/(poly(methyl methacrylate) composite latex particles investigated by TEM and NMR,” Acta Polymerica 1999, 50, 347-362; K. Landfester, N. Bechthold, S. Förster and M. Antonietti, “Evidence for the preservation of the particle identity in miniemulsion polymerization,” Macromol. Rapid Commun. 1999, 20, 81-84; K. Landfester, N. Bechthold, F. Tiarks and M. Antonietti, “Miniemulsion polymerization with cationic and nonionic surfactants: A very efficient use of surfactants for heterophase polymerization” Macromolecules 1999, 32, 2679-2683; K. Landfester, N. Bechthold, F. Tiarks and M. Antonietti, “Formulation and stability mechanisms of polymerizable miniemulsions,” Macromolecules 1999, 32, 5222-5228; G. Baskar, K. Landfester and M. Antonietti, “Comb-like polymers with octadecyl side chain and carboxyl functional sites: Scope for efficient use in miniemulsion polymerization,” Macromolecules 2000, 33, 9228-9232; N. Bechthold, F. Tiarks, M. Willert, K. Landfester and M. Antonietti, “Miniemulsion polymerization: Applications and new materials” Macromol. Symp. 2000, 151, 549-555; N. Bechthold and K. Landfester: “Kinetics of miniemulsion polymerization as revealed by calorimetry,” Macromolecules 2000, 33, 4682-4689; B. M. Budhlall, K. Landfester, D. Nagy, E. D. Sudol, V. L. Dimonie, D. Sagl, A. Klein and M. S. El-Aasser, “Characterization of partially hydrolyzed poly(vinyl alcohol). I. Sequence distribution via H-1 and C-13-NMR and a reversed-phased gradient elution HPLC technique,” Macromol. Symp. 2000, 155, 63-84; D. Columbie, K. Landfester, E. D. Sudol and M. S. El-Aasser, “Competitive adsorption of the anionic surfactant Triton X-405 on PS latex particles,” Langmuir 2000, 16, 7905-7913; S. Kirsch, A. Pfau, K. Landfester, O. Shaffer and M. S. El-Aasser, “Particle morphology of carboxylated poly-(n-butyl acrylate)/poly(methyl methacrylate) composite latex particles,” Macromol. Symp. 2000, 151, 413-418; K. Landfester, F. Tiarks, H.-P. Hentze and M. Antonietti, “Polyaddition in miniemulsions: A new route to polymer dispersions,” Macromol. Chem. Phys. 2000, 201, 1-5; K. Landfester, “Recent developments in miniemulsions—Formation and stability mechanisms,” Macromol. Symp. 2000, 150, 171-178; K. Landfester, M. Willert and M. Antonietti, “Preparation of polymer particles in non-aqueous direct and inverse miniemulsions,” Macromolecules 2000, 33, 2370-2376; K. Landfester and M. Antonietti, “The polymerization of acrylonitrile in miniemulsions: ‘Crumpled latex particles’ or polymer nanocrystals,” Macromol. Rapid Comm. 2000, 21, 820-824; B. z. Putlitz, K. Landfester, S. Förster and M. Antonietti, “Vesicle forming, single tail hydrocarbon surfactants with sulfonium-headgroup,” Langmuir 2000, 16, 3003-3005; B. z. Putlitz, H.-P. Hentze, K. Landfester and M. Antonietti, “New cationic surfactants with sulfonium-headgroup,” Langmuir 2000, 16, 3214-3220; J. Rottstegge, K. Landfester, M. Wilhelm, C. Heldmann and H. W. Spiess, “Different types of water in film formation process of latex dispersions as detected by solid-state nuclear magnetic resonance spectroscopy,” Colloid Polym. Sci. 2000, 278, 236-244; M. Antonietti and K. Landfester, “Single molecule chemistry with polymers and colloids: A way to handle complex reactions and physical processes?” ChemPhysChem 2001, 2, 207-210; K. Landfester and H.-P. Hentze, “Heterophase polymerization in inverse systems,” in Reactions and Synthesis in Surfactant Systems, J. Texter, ed.; Marcel Dekker, Inc., New York, 2001, pp 471-499; K. Landfester, “Polyreactions in miniemulsions,” Macromol. Rapid Comm. 2001, 896-936; K. Landfester, “The generation of nanoparticles in miniemulsion,” Adv. Mater. 2001, 10, 765-768; K. Landfester, “Chemie—Rezeptionsgeschichte” in Der Neue Pauly—Enzyklopädie der Antik, Verlag J. B. Metzler, Stuttgart, 2001, vol. 15; B. z. Putlitz, K. Landfester, H. Fischer and M. Antonietti, “The generation of ‘armored latexes’ and hollow inorganic shells made of clay sheets by templating cationic miniemulsions and latexes,” Adv. Mater. 2001, 13, 500-503; F. Tiarks, K. Landfester and M. Antonietti, “Preparation of polymeric nanocapsules by miniemulsion polymerization,” Langmuir 2001, 17, 908-917; F. Tiarks, K. Landfester and M. Antonietti, “Encapsulation of carbon black by miniemulsion polymerization,” Macromol. Chem. Phys. 2001, 202, 51-60; F. Tiarks, K. Landfester and M. Antonietti, “One-step preparation of polyurethane dispersions by miniemulsion polyaddition,” J. Polym. Sci. Polym. Chem. Ed. 2001, 39, 2520-2524; F. Tiarks, K. Landfester and M. Antonietti, “Silica nanoparticles as surfactants and fillers for latexes made by miniemulsion polymerization,” Langmuir 2001, 17, 5775-5780.

The encapsulated metal-based compounds may be produced in a size of about 1 nm to 500 nm, or in the form of microparticles having sizes from about 5 nm to 5 μm. Metal-based compounds may be further encapsulated in mini- or micro-emulsions of suitable polymers. The term mini- or micro-emulsion can be understood as dispersions comprising an aqueous phase, an oil phase, and surface active substances. Such emulsions may comprise suitable oils, water, one or several surfactants, optionally one or several co-surfactants, and one or several hydrophobic substances. Mini-emulsions may comprise aqueous emulsions of monomers, oligomers or other pre-polymeric reactants stabilised by surfactants, which may be easily polymerized, and wherein the particle size of the emulgated droplets is between about 10 nm to 500 nm or larger.

Furthermore, mini-emulsions of encapsulated metal-based compounds can be made from non-aqueous media, for example, formamide, glycol, or non-polar solvents. In principle, pre-polymeric reactants may be selected from thermosets, thermoplastics, plastics, synthetic rubbers, extrudable polymers, injection molding polymers, moldable polymers, and the like or mixtures thereof, including pre-polymeric reactants from which poly(meth)acrylics can be used.

Examples of suitable polymers for encapsulating the metal-based compounds can include, but are not limited to, homopolymers or copolymers of aliphatic or aromatic polyolefins such as polyethylene, polypropylene, polybutene, polyisobutene, polypentene; polybutadiene; polyvinyls such as polyvinyl chloride or polyvinyl alcohol, poly(meth)acrylic acid, polymethylmethacrylate (PMMA), polyacrylocyano acrylate; polyacrylonitril, polyamide, polyester, polyurethane, polystyrene, polytetrafluoroethylene; biopolymers such as collagen, albumin, gelatine, hyaluronic acid, starch, celluloses such as methylcellulose, hydroxypropyl cellulose, hydroxypropyl methylcellulose, carboxymethylcellulose phthalate; casein, dextranes, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids; polyethylene vinyl acetate, silicones; poly(ester urethanes), poly(ether urethanes), poly(ester ureas), polyethers such as polyethylene oxide, polypropylene oxide, pluronics, polytetramethylene glycol; polyvinylpyrrolidone, poly(vinyl acetate phthalate), shellac, and combinations of these homopolymers or copolymers.

Further encapsulating materials that may be used can include poly(meth)acrylate, unsaturated polyester, saturated polyester, polyolefines such as polyethylene, polypropylene, polybutylene, alkyd resins, epoxy-polymers or resins, polyamide, polyimide, polyetherimide, polyamideimide, polyesterimide, polyesteramideimide, polyurethane, polycarbonate, polystyrene, polyphenole, polyvinylester, polysilicone, polyacetale, cellulosic acetate, polyvinylchloride, polyvinylacetate, polyvinylalcohol, polysulfone, polyphenylsulfone, polyethersulfone, polyketone, polyetherketone, polybenzimidazole, polybenzoxazole, polybenzthiazole, polyfluorocarbons, polyphenylenether, polyarylate, cyanatoester-polymere, and mixtures or copolymers of any of the foregoing are preferred.

In certain exemplary embodiments of the present invention, the polymers for encapsulating the metal-based compounds may be selected from mono(meth)acrylate-, di(meth)acrylate-, tri(meth)acrylate-, tetra-acrylate and pentaacrylate-based poly(meth)acrylates. Examples of suitable mono(meth)acrylates include hydroxyethyl acrylate, hydroxyethyl methacrylate, hydroxypropyl methacrylate, hydroxypropyl acrylate, 3-chloro-2-hydroxypropyl acrylate, 3-chloro-2-hydroxypropyl methacrylate, 2,2-dimethylhydroxypropyl acrylate, 5-hydroxypentyl acrylate, diethylene glycol monoacrylate, trimethylolpropane monoacrylate, pentaerythritol monoacrylate, 2,2-dimethyl-3-hydroxypropyl acrylate, 5-hydroxypentyl methacrylate, diethylene glycol monomethacrylate, trimethylolpropane monomethacrylate, pentaerythritol monomethacrylate, hydroxy-methylated N-(1,1-dimethyl-3-oxobutyl)acrylamide, N-methylolacrylamide, N-methylolmethacrylamide, N-ethyl-N-methylolmethacrylamide, N-ethyl-N-methylolacrylamide, N,N-dimethylol-acrylamide, N-ethanolacrylamide, N-propanolacrylamide, N-methylolacrylamide, glycidyl acrylate, and glycidyl methacrylate, methyl acrylate, ethyl acrylate, propyl acrylate, butyl acrylate, amyl acrylate, ethylhexyl acrylate, octyl acrylate, t-octyl acrylate, 2-methoxyethyl acrylate, 2-butoxyethyl acrylate, 2-phenoxyethyl acrylate, chloroethyl acrylate, cyanoethyl acrylate, dimethylaminoethyl acrylate, benzyl acrylate, methoxybenzyl acrylate, furfuryl acrylate, tetrahydrofurfuryl acrylate and phenyl acrylate; di(meth)acrylates may be selected from 2,2-bis(4-methacryloxyphenyl)propane, 1,2-butanediol-diacrylate, 1,4-butanediol-diacrylate, 1,4-butanediol-dimethacrylate, 1,4-cyclohexanediol-dimethacrylate, 1,10-decanediol-dimethacrylate, diethylene-glycol-diacrylate, dipropyleneglycol-diacrylate, dimethyl-propanediol-dimethacrylate, triethyleneglycol-dimethacrylate, tetraethyleneglycol-dimethacrylate, 1,6-hexanediol-diacrylate, Neopentylglycol-diacrylate, polyethylene-glycol-dimethacrylate, tripropyleneglycol-diacrylate, 2,2-bis[4-(2-acryloxyethoxy)-phenyl]propane, 2,2-bis[4-(2-hydroxy-3-methacryloxypropoxy)phenyl]propane, bis(2-methacryloxyethyl)N,N-1,9-nonylene-biscarbamate, 1,4-cycloheanedimethanol-dimethacrylate, and diacrylic urethane oligomers; tri(meth)acrylates may be selected from tris(2-hydroxyethyl)isocyanurate-trimethacrylate, tris(2-hydroxyethyl)isocyanurate-triacrylate, trimethylolpropane-trimethacrylate, trimethylolpropane-triacrylate or pentaerythritol-triacrylate; tetra(meth)acrylates may be selected from pentaerythritol-tetraacrylate, di-trimethylopropan-tetraacrylate, or ethoxylated pentaerythritol-tetraacrylate; suitable penta(meth)acrylates may be selected from dipentaerythritol-pentaacrylate or pentaacrylate-esters; as well as mixtures, copolymers and combinations of any of the foregoing.

In medical applications, biopolymers or acrylics may be preferably selected as polymers for encapsulating the metal-based compounds.

Encapsulating polymer reactants may be selected from polymerisable monomers, oligomers or elastomers such as polybutadiene, polyisobutylene, polyisoprene, poly(styrene-butadiene-styrene), polyurethanes, polychloroprene, or silicone, and mixtures, copolymers or combinations of any of the foregoing. The metal-based compounds may be encapsulated in elastomeric polymers solely or in mixtures of thermoplastic and elastomeric polymers or in a sequence of shells/layers alternating between thermoplastic and elastomeric polymer shells.

The polymerization reaction for encapsulating the metal-based compounds may be any suitable conventional polymerization reaction, for example, a radical or non-radical polymerization, enzymatical or non-enzymatical polymerization, including a poly-condensation reaction. The emulsions, dispersions or suspensions used may be in the form of aqueous, non-aqueous, polar or unpolar systems. By adding suitable surfactants, the amount and size of the emulgated or dispersed droplets can be adjusted as required. The surfactants may be anionic, cationic, zwitter-ionic or non-ionic surfactants or any combinations thereof. Preferred anionic surfactants may include, but are not limited to, soaps, alkylbenzolsulphonates, alkansulphonates, olefinsulphonates, alkyethersulphonates, glycerinethersulphonates, α-methylestersulphonates, sulphonated fatty acids, alkylsulphates, fatty alcohol ether sulphates, glycerine ether sulphates, fatty acid ether sulphates, hydroxyl mixed ether sulphates, monoglyceride(ether)sulphates, fatty acid amide(ether)sulphates, mono- and di-alkylsulfosuccinates, mono- and dialkylsulfosuccinamates, sulfotriglycerides, amidsoaps, ethercarboxylicacid and their salts, fatty acid isothionates, fatty acid arcosinates, fatty acid taurides, N-acylaminoacid such as acyllactylates, acyltartrates, acylglutamates and acylaspartates, alkyoligoglucosidsulfates, protein fatty acid condensates, including plant derived products based on wheat; and alky(ether)phosphates.

Cationic surfactants suitable for encapsulation reactions in certain exemplary embodiments of the present invention may be selected from the group of quaternary ammonium compounds such as dimethyldistearylammoniumchloride, Stepantex® VL 90 (Stepan), esterquats, particularly quaternised fatty acid trialkanolaminester salts, salts of long-chain primary amines, quaternary ammonium compounds like hexadecyltrimethylammoniumchloride (CTMA-Cl), Dehyquart® A (cetrimonium-chloride, Cognis), or Dehyquart® LDB 50 (lauryldimethylbenzylammoniumchloride, Cognis).

The metal-based compounds, which may be in the form of a metal-based sol, can be added before or during the start of the polymerization reaction, and may be provided as a dispersion, emulsion, suspension or solid solution, or solution of the metal-based compounds in a suitable solvent or solvent mixture, or any mixtures thereof. The encapsulation process can require the polymerization reaction, optionally with the use of initiators, starters or catalysts, wherein an in-situ encapsulation of the metal-based compounds in the polymer produced by the polymerization in polymer capsules, spheroids or droplets is provided. The solids content of the metal-based compounds in such encapsulation mixtures may be selected such that the solids content in the polymer capsules, spheroids or droplets can be about 10 weight % to 80 weight % of metal-based compound within the polymer particles.

Optionally, additional metal-based precursor compounds may be added after completion of the polymerization reaction, either in solid form or in a liquid form, to bind on or coat the encapsulated metal-based compounds. In an alternative exemplary embodiment of the present invention, the metal-based compounds as described above may be coated onto polymeric particles, polymer spheres, polymer bubbles or polymeric shells. The metal-based compounds can be selected from those compounds which are able to bind to the polymer spheroids or droplets covalently or non-covalently. For coating polymer particles, polymer particles produced in liquid media polymerization processes may be used, and the methods described above for encapsulating metal-based compounds, e.g., by emulsion polymerization, can also be used to produce polymer particles in suspension, emulsion or dispersion, which may be subsequently coated with the metal-based compounds, typically by adding the metal-based compounds to the polymerized reaction mixture.

The term “encapsulated metal-based compounds” may be understood to include polymer particles coated with metal-based compounds.

The droplet size of the polymers and the solids content of metal-based compounds may be selected such that the solids content of the encapsulated metal-based compounds and/or metal coated polymer particles is in the range of about 5 weight % to 60 weight % of the polymerization reaction mass.

In one exemplary embodiment of the present invention, the in-situ encapsulation of the metal-based compounds during the polymerization may be repeated by addition of further monomers, oligomers or pre-polymeric agents after completion of the first polymerization/encapsulation step. By providing at least one similar repeated step, multilayer coated polymer capsules may be produced. Also, metal-based compounds bound to polymer spheroids or droplets may be encapsulated by subsequently adding monomers, oligomers or pre-polymeric reactants to overcoat the metal-based compounds with a polymer capsule. Repetition of such process steps can provide multilayered polymer capsules comprising the metal-based compound.

Any of these encapsulation steps may be combined with each other. In a particular exemplary embodiment of the present invention, polymer encapsulated metal-based compounds may be further encapsulated with elastomeric compounds, so that polymer capsules having an outer elastomer shell are produced.

In further exemplary embodiments of the present invention, polymer encapsulated metal-based compounds may be further encapsulate in vesicles, liposomes or micelles, or overcoatings. Suitable surfactants for this purpose may include the surfactants described above, and compounds having hydrophobic groups which may include hydrocarbon residues or silicon residues, for example, polysiloxane chains, hydrocarbon based monomers, oligomers and polymers, or lipids or phosphorlipids, or any combinations thereof, particularly glycerylester such as phosphatidylethanolamine, phosphatidylcholine, polyglycolide, polylactide, polymethacrylate, polyvinylbuthylether, polystyrene, polycyclopentadienylmethylnorbornene, polypropylene, polyethylene, polyisobutylene, polysiloxane, or any other type of surfactant.

Furthermore, depending on the polymeric shell, surfactants for encapsulating the polymer encapsulated metal-based compounds in vesicles, overcoats and the like may be selected from hydrophilic surfactants or surfactants having hydrophilic residues or hydrophilic polymers such as polystyrensulfonicacid, poly-N-alkylvinylpyridiniumhalogenide, poly(meth)acrylic acid, polyaminoacids, poly-N-vinylpyrrolidone, polyhydroxyethylmethacrylate, polyvinylether, polyethylenglycol, polypropylenoxide, polysaccharides such as agarose, dextrane, starch, cellulose, amylase, amylopektine or polyethylenglycole, or polyethylennimine of a suitable molecular weight. Also, mixtures from hydrophobic or hydrophilic polymer materials or lipid polymer compounds may be used for encapsulating the polymer capsulated metal-based compounds in vesicles or for further over-coating the polymer encapsulating metal-based compounds.

Additionally, the encapsulated metal-based compounds may be chemically modified by functionalization with suitable linker groups or coatings which are capable to react with the sol/gel forming components. For example, they may be functionalized with organosilane compounds or organo-functional silanes. Such compounds for modification of the polymer encapsulating metal-based compounds are further described in the below sol/gel component section.

The incorporation of polymer-encapsulated metal-based compounds into the materials produced in accordance with exemplary embodiments of the present invention can be regarded as a specific form of a filler. The particle size and particle size distribution of the polymer-encapsulated metal-based compounds in dispersed or suspended form may correspond to the particle size and particle size distribution of the particles of finished polymer-encapsulated metal-based compounds, and they can have a significant influence on the pore sizes of the material produced. The polymer-encapsulated metal-based compounds can be characterized by dynamic light scattering methods to determine their average particle size and monodispersity.

Sol/Gel Forming Components

The polymer encapsulated metal-based compounds may be combined with a sol before subsequently being converted into a solid metal containing composite material.

The sol utilized in the exemplary embodiments of the present invention can be prepared from any type of sol/gel forming components in a conventional manner. uitable components and/or sols may be selected for combination with the polymer encapsulated metal-based compounds.

The sol/gel forming components may be selected from alkoxides, oxides, acetates, nitrates of various metals, e.g., silicon, aluminum, boron, magnesium, zirconium, titanium, alkaline metals, alkaline earth metals, or transition metals, and from platinum, molybdenum, iridium, tantalum, bismuth, tungsten, vanadium, cobalt, hafnium, niobium, chromium, manganese, rhenium, iron, gold, silver, copper, ruthenium, rhodium, palladium, osmium, lanthanum and lanthanides, as well as combinations thereof.

In some exemplary embodiments of the present invention, the sol/gel forming components can be metal oxides, metal carbides, metal nitrides, metal oxynitrides, metal carbonitrides, metal oxycarbides, metal oxynitrides, or metal oxycarbonitrides of the above mentioned metals, or any combinations thereof. These compounds, which may be in the form of colloidal particles, can be reacted with oxygen-containing compounds, e.g. alkoxides to form a sol/gel, or may be added as fillers if not in colloidal form. Where the sol is formed from metal-based compounds such as those mentioned above, at least a part of these sol forming compounds may be encapsulated in a polymeric shell, i.e., the encapsulated metal-based compound and the sol forming compound may be substantially the same.

In other exemplary embodiments of the present invention, the sols may be derived from at least one sol/gel forming component such as alkoxides, metal alkoxides, colloidal particles, particularly metal oxides, and the like. The metal alkoxides that may be used as sol/gel forming components may be conventional chemical compounds that can be used in a variety of applications. These compounds can have the general formula M(OR)ₓ, wherein M is any metal from a metal alkoxide which, e.g., may hydrolyze and polymerize in the presence of water. R is an alkyl radical of 1 to 30 carbon atoms, which may be straight chained or branched, and x has a value equivalent to the metal ion valence. Metal alkoxides such as Si(OR)₄, Ti(OR)₄, Al(OR)₃, Zr(OR)₃ and Sn(OR)₄ may be used. Specifically, R can be the methyl, straight-chain, or branched ethyl, propyl or butyl radical. Further examples of suitable metal alkoxides can include Ti(isopropoxy)₄, Al(isopropoxy)₃, Al(sec-butoxy)₃, Zr(n-butoxy)₄ and Zr(n-propoxy)₄.

Sols can be made from silicon alkoxides like tetraalkoxysilanes, wherein the alkoxy may be branched or straight chained and may contain about 1 to 25 carbon atoms, e.g., tetramethoxysilane (TMOS), tetraethoxysilane (TEOS) or tetra-n-propoxysilane, as well as oligomeric forms thereof. Also suitable are alkylalkoxysilanes, wherein alkoxy is defined as above and alkyl may be a substituted or unsubstituted, branched or straight chain alkyl having about 1 to 25 carbon atoms, e.g., methyltrimethoxysilane (MTMOS), methyltriethoxysilane, ethyltriethoxysilane, ethyltrimethoxysilane, methyltripropoxysilane, methyltributoxysilane, propyltrimethoxysilane, propyltriethoxysilane, isobutyltriethoxysilane, isobutyltrimethoxy-silane, octyltriethoxysilane, octyltrimethoxysilane, which is commercially available from Degussa AG, Germany, methacryloxydecyltrimethoxysilane (MDTMS); aryltrialkoxy-silanes such as phenyltrimethoxysilane (PTMOS), phenyltriethoxysilane, which is commercially available from Degussa AG, Germany; phenyltripropoxysilane, and phenyltributoxysilane, phenyl-tri-(3-glycidyloxy)-silane-oxide (TGPSO), 3-aminopropyl-trimethoxysilane, 3-aminopropyl-triethoxysilane, 2-aminoethyl-3-aminopropyl-trimethoxysilane, triaminofunctional propyltrimethoxysilane (Dynasylan® TRIAMO, available from Degussa AG, Germany), N-(n-butyl)-3-aminopropyltrimethoxysilane, 3-aminopropylmethyl-diethoxysilane, 3-glycidyloxypropyltrimethoxysilane, 3-glycidyloxypropyltriethoxy-silane, vinyltrimethoxysilane, vinyltriethoxysilane, 3-mercaptopropyltrimethoxy-silane, Bisphenol-A-glycidylsilanes; (meth)acrylsilanes, phenylsilanes, oligomeric or polymeric silanes, epoxysilanes; fluoroalkylsilanes such as fluoroalkyltrimethoxysilanes, fluoroalkyltriethoxysilanes with a partially or fully fluorinated, straight chain or branched fluoroalkyl residue of about 1 to 20 carbon atoms, e.g., tridecafluoro-1,1,2,2-tetrahydrooctyltriethoxysilane and modified reactive flouroalkylsiloxanes which are available from Degussa AG under the trademarks Dynasylan® F8800 and F8815; as well as any mixtures of the foregoing.

In another exemplary embodiment of the present invention, sol may be prepared from carbon-based nanoparticles and alkaline metal salts, e.g. acetates, as well as acids, such as phosphorous acids, pentoxides, phosphates, or organo phosphorous compounds such as alkyl phosphonic acids. Further substances that may be used to form sols include calcium acetate, phosphorous acid, P₂O₅, as well as triethyl phosphite as a sol in ethanediol, whereby biodegradable composites can be prepared from carbon-based nanoparticles and physiologically acceptable inorganic components. By varying the stoichiometric Ca/P-ratio, the degeneration rate of such composites can be adjusted. The molar ratio of Ca to P can be about 0.1 to 10, or preferably about 1 to 3.

In certain exemplary embodiments of the present invention, the sols can be prepared from colloidal solutions, which may comprise carbon-based nanoparticles, preferably in solution, dispersion or suspension in polar or nonpolar solvents, including aqueous solvents as well as cationically or anionically polymerizable polymers as precursors, such as alginate. By addition of suitable coagulators, e.g., inorganic or organic acids or bases, in particular acetates and diacetates, carbon containing composite materials can be produced by precipitation or gel formation. Optionally, further particles can be added to adjust the properties of the resultant material. These particles may comprise, e.g., metals, metal oxides, metal carbides, or mixtures thereof, as well as metal acetates or diacetates.

The sol/gel components used in the sols may also comprise colloidal metal oxides, preferably those colloidal metal oxides which are stable long enough to be able to combine them with the other sol/gel components and the polymer-encapsulated metal-based compounds. Such colloidal metal oxides may include, but are not limited to, SiO₂, Al₂O₃, MgO, ZrO₂, TiO₂, SnO₂, ZrSiO₄, B₂O₃, La₂O₃, Sb₂O₅ and ZrO(NO₃)₂. SiO₂, Al₂O₃, ZrSiO₄ and ZrO₂ may be preferably selected. Further examples of the at least one sol/gel forming component include aluminumhydroxide sols or gels, aluminumtri-sec-butylat, AlOOH-gels and the like.

Some of these colloidal sols may be acidic in the sol form and, therefore, when used during hydrolysis, it may not be necessary to add additional acid to the hydrolysis medium. These colloidal sols can also be prepared by a variety of methods. For example, titania sols having a particle size in the range of about 5 to 150 nm can be prepared by the acidic hydrolysis of titanium tetrachloride, by peptizing hydrous TiO₂ with tartaric acid, and by peptizing ammonia washed Ti(SO₄)₂ with hydrochloric acid. Such processes are descrbed, for example, by Weiser in Inorganic Colloidal Chemistry, Vol. 2, p. 281 (1935). In order to preclude the incorporation of contaminants in the sols, the alkyl orthoesters of the metals can be hydrolyzed in an acid pH range of about 1 to 3, in the presence of a water miscible solvent, wherein the colloid is present in the dispersion in an amount of about 0.1 to 10 weight percent.

In certain exemplary embodiments of the present invention, the sols can be made of sol/gel forming components such as metal halides of the metals as mentioned above, which are reacted with oxygen functionalized polymer-encapsulated metal-based compounds to form the desired sol. In this case, the sol/gel forming components may be oxygen-containing compounds, e.g., alkoxides, ethers, alcohols or acetates, which can be reacted with suitably functionalized polymer-encapsulated metal-based compounds. However, normally the polymer-encapsulated metal-based compounds can be dispersed into the sol by suitable blending methods, or a metal-based sol may be incorporated in a polymerization process, wherein at least a part of the metal-based sol compounds may be encapsulated by the polymer.

Where the sol is formed by a hydrolytic sol/gel-process, the molar ratio of the added water and the sol/gel forming component, such as alkoxides, oxides, acetates, nitrides or combinations thereof, may be in the range of about 0.001 to 100, or preferably from about 0.1 to 80, or more preferably from aout 0.2 to 30.

In a typical hydrolytric sol/gel processing procedure which can be used with the exemplary embodiments of the present invention, the sol/gel components are blended with the (optionally chemically modified) polymer-encapsulated metal-based compounds in the presence of water. Optionally, further solvents or mixtures thereof, and/or further additives may be added, such as surfactants, fillers and the like, as described in more detail hereinafter. Further additives such as crosslinkers may also be added as catalysts for controlling the hydrolysis rate of the sol or for controlling the crosslinking rate. Such catalysts are also described in further detail hereinbelow. Such processing is similar to conventional sol/gel processing.

Non-hydrolytic sols may be made in a manner similar to that described above, but likely essentially in the absence of water.

When the sol is formed by a non-hydrolytic sol/gel-process or by chemically linking the components with a linker, the molar ratio of the halide and the oxygen-containing compound may be in the range of about 0.001 to 100, or preferably from about 0.1 to 140, or more preferably from about 0.1 to 100, or even more preferably from about 0.2 to 80.

In nonhydrolytic sol/gel processes, the use of metal alkoxides and carboxylic acids and their derivatives, or carboxylic acid functionalized polymer-encapsulated metal-based compounds, may also be suitable. Suitable carboxylic acids include acetic acid, acetoacetic acid, formic acid, maleic acid, crotonic acid, or succinic acid.

Non-hydrolytic sol/gel processing in the absence of water may be accomplished by reacting alkylsilanes or metal alkoxides with anhydrous organic acids, acid anhydrides or acid esters, or the like. Acids and their derivatives may be suitable as sol/gel components or for modifying and/or functionalizing the polymer-encapsulated metal-based compounds.

In certain exemplary embodiments of the present invention, the sol may also be formed from at least one sol/gel forming component in a nonhydrous sol/gel processing, and the reactants can be selected from anhydrous organic acids, acid anhydrides or acid esters like formic acid, acetic acid, acetoacetic acid, succinic acid maleic acid, crotonic acid, acrylic acid, methacrylic acid, partially or fully fluorinated carboxylic acids, their anhydrides and esters, e.g. methyl- or ethylesters, or any mixtures of the foregoing. It may be preferred to use acid anhydrides in admixture with anhydrous alcohols, wherein the molar ratio of these components determines the amount of residual acetoxy groups at the silicon atom of the alkylsilane employed.

Typically, according to the degree of cross-linking desired in the resulting sol or combination of sol and polymer-encapsulated metal-based compounds, either acidic or basic catalysts may be applied, particularly in hydrolytic sol/gel processes. Suitable inorganic acids may include, for example, hydrochloric acid, sulfuric acid, phosphoric acid, nitric acid as well as diluted hydrofluoric acid. Suitable bases include, for example, sodium hydroxide, ammonia and carbonate as well as organic amines. Suitable catalysts in non-hydrolytic sol/gel processes can include anhydrous halide compounds, for example, BCl₃, NH₃, AlCl₃, TiCl₃ or mixtures thereof.

To affect the hydrolysis in hydrolytic sol/gel processing steps of the present invention, the addition of solvents may be used, including water-miscible solvents such as water-miscible alcohols or mixtures thereof. Alcohols such as methanol, ethanol, n-propanol, isopropanol, n-butanol, isobutanol, t-butanol and lower molecular weight ether alcohols such as ethylene glycol monomethyl ether may be used. Small amounts of non-water-miscible solvents such as toluene may also be advantageously used in certain exemplary embodiments of the present invention. These solvents can also be used in polymer encapsulation reactions such as those described above.

Additives

The properties of the composite materials produced in accordance with certain exemplary embodiments of the present invention, e.g., resistance to mechanical stress, electrical conductivity, impact strength or optical properties, can be varied by application of suitable amounts of additives, particularly with the addition of organic polymer materials. Further additives can be added to the sol or the combination, which do not react with the components thereof.

Examples of suitable additives include fillers, pore-forming agents, metals and metal powders, and the like. Examples of inorganic additives and fillers can include silicon oxides and aluminum oxides, aluminosilicates, zeolites, zirconium oxides, titanium oxides, talc, graphite, carbon black, fullerenes, clay materials, phyllosilicates, silicides, nitrides, metal powders, in particular those of catalytically active transition metals such as copper, gold, silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum.

By means of such additives, it is possible to further vary and adjust the mechanical, optical and thermal properties of the resultant material. The use of such additives may be particularly suitable for producing tailor-made coatings having desired properties.

Further suitable additives can include fillers, crosslinkers, plasticizers, lubricants, flame resistants, glass or glass fibers, carbon fibers, cotton, fabrics, metal powders, metal compounds, silicon, silicon oxides, zeolites, titan oxides, zirconium oxides, aluminum oxides, aluminum silicates, talcum, graphite, soot, phyllosilicates and the like.

In certain exemplary embodiments of the present invention, the sol or combination network may be further modified by the addition of at least one crosslinking agent to the sol, the polymer-encapsulated metal-based compounds or the combination. The crosslinking agent may comprise, for example, isocyanates, silanes, diols, di-carboxylic acids, (meth)acrylates, for example 2-hydroxyethyl methacrylate, propyltrimethoxysilane, 3-(trimethylsilyl)propyl methacrylate, isophoron diisocyanate, polyols, glycerine, and the like. Biocompatible crosslinkers such as glycerine, diethylentriaminoisocyanate and 1,6-diisocyanatohexane may be used, wherein the sol/gel is converted into the solid material at relatively low temperatures, e.g. below about 100° C. The use of suitable crosslinkers in combination with the incorporation of polymer-encapsulated metal-based compounds may be used to form composite materials having an anisotropic porosity, i.e., a gradient of the pore size through the composite material. The anisotropic porosity may be further influenced by fillers, as discussed above and below hereinafter.

Fillers can be used to modify the size and the degree of porosity. In some certain exemplary embodiments of the present invention, non-polymeric fillers may be preferred. Non-polymeric fillers can be any substance which can be removed or degraded, for example, by thermal treatment or other conditions, without adversely affecting the material properties. Some fillers might be resolved in a suitable solvent and can be removed in this manner from the material. Furthermore, non-polymeric fillers, which can be converted into soluble substances under chosen thermal conditions, can also be used. These non-polymeric fillers may comprise, for example, anionic, cationic or non-ionic surfactants, which can be removed or degraded under thermal conditions.

In another exemplary embodiment of the present invention, the fillers may comprise inorganic metal salts, particularly salts from alkaline and/or alkaline earth metals, including alkaline or alkaline earth metal carbonates, sulfates, sulfites, nitrates, nitrites, phosphates, phosphites, halides, sulfides, oxides, or mixtures thereof. Other suitable fillers include organic metal salts, e.g., alkaline or alkaline earth and/or transition metal salts, including formiates, acetates, propionates, malates, maleates, oxalates, tartrates, citrates, benzoates, salicylates, phtalates, stearates, phenolates, sulfonates, or amines, as well as mixtures thereof.

In yet another exemplary embodiment of the present invention, polymeric fillers may be applied. Suitable polymeric fillers can be those as mentioned above as encapsulation polymers, particularly those having the form of spheres or capsules. Saturated, linear or branched aliphatic hydrocarbons may also be used, and they may be homo- or copolymers. Polyolefins such as polyethylene, polypropylene, polybutene, polyisobutene, polypentene as well as copolymers thereof and mixtures thereof may be preferably used. Polymeric fillers may also comprise polymer particles formed of methacrylates or polystearine, as well as electrically conducting polymers such as polyacetylenes, polyanilines, poly(ethylenedioxythiophenes), polydialkylfluorenes, polythiophenes or polypyrroles, which may be used to provide electrically conductive materials.

In some or many of the above-mentioned procedures, the use of soluble fillers can be combined with addition of polymeric fillers, wherein the fillers may be volatile under thermal processing conditions or may be converted into volatile compounds during thermal treatment. In this way the pores formed by the polymeric fillers can be combined with the pores formed by the other fillers to achieve an isotropic or anisotropic pore distribution.

Suitable particle sizes of the non-polymeric fillers can be determined based on the desired porosity and/or size of the pores of the resulting composite material.

Porosity in the resultant composite materials can be produced by treatment processes such as those described in German Patent Publication DE 103 35 131 and in PCT Application No. PCT/EP04/00077.

Further additives that may be used in exemplary embodiments of the present invention may include, e.g., drying-control chemical additives such as glycerol, DMF, DMSO, or any other suitable high boiling point or viscous liquid that can be suitable for controlling the conversion of the sols to gels and solid composites.

Solvents that can be used for the removal of the fillers after thermal treatment of the material may include, for example, (hot) water, diluted or concentrated inorganic or organic acids, bases, and the like. Suitable inorganic acids can include, for example, hydrochloric acid, sulfuric acid, phosphoric acid, nitric acid, as well as diluted hydrofluoric acid. Suitable bases can include, for example, sodium hydroxide, ammonia, carbonate, as well as organic amines. Suitable organic acids can include, for example, formic acid, acetic acid, trichloromethane acid, trifluoromethane acid, citric acid, tartaric acid, oxalic acid, and mixtures thereof.

In certain exemplary embodiments of the present invention, coatings of the inventive composite materials may be applied as a liquid solution or dispersion or suspension of the combination in a suitable solvent or solvent mixture, with subsequent drying or evaporation of the solvent. Suitable solvents may comprise, for example, methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofurane, trimethyl hexanol, phenol, benzene, toluene, xylene; as well as water, any of which may be mixed with dispersants, surfactants, or other additives, and mixtures of the above-named substances.

Any of the above- and below-mentioned solvents can also be used in the sol/gel process.

Solvents may also comprise one or several organic solvents such as ethanol, isopropanol, n-propanol, dipropylene glycol methyl ether and butoxyisopropanol (1,2-propylene glycol-n-butyl ether), tetrahydrofurane, phenol, benzene, toluene, xylene, preferably ethanol, isopropanol, n-propanol and/or dipropylene glycol methyl ether, methylethylketone, wherein isopropanol and/or n-propanol may be preferably selected.

The fillers can be partly or completely removed from the resultant composite material, depending on the nature and time of treatment with the solvent. A complete removal of the filler may be preferable in certain exemplary embodiments of the present invention.

Conversion

The combination of sol and polymer-encapsulated metal-based compounds can be converted into a solid metal-containing composite material. Conversion of the sol/combination into gel may be accomplished by, e.g., aging, curing, raising of pH, evaporation of solvent, or any other conventional methods.

The sol/combination may be first converted into a gel and subsequently converted into a solid composite material, or the sol/combination may be directly converted into the composite material, particularly where the components used can produce polymeric glassy composites, aerogels or xerogels, and further wherein they may be produced at room temperature.

The conversion step can be achieved by drying the sol or gel. In certain exemplary embodiments of the present invention, this drying step may be a thermal treatment of the sol or gel, which further may optionally be a pyrolysis or carbonization step, in the range of about −200 C to 3500 C, or preferably in the range of about −100° C. to 2500° C., or more preferably in the range of about −50° C. to 1500° C., even more prefrably about 0° C. to 1000° C., or yet even more preferably about 50° C. to 800° C., or at approximatey room temperature. Thermal treatment may also be performed by laser applications, e.g. by selective laser sintering (SLS).

The conversion of the sol/combination into the solid material can be performed under various conditions. The conversion can be performed in different atmospheres, e.g. inert atmospheres such as nitrogen, SF₆, or noble gases such as argon, or any mixtures thereof, or it may be performed in an oxidizing atmosphere such as oxygen, carbon monoxide, carbon dioxide, or nitrogen oxide, or any mixtures thereof. Furthermore, an inert atmosphere may be blended with reactive gases, e.g., hydrogen, ammonia, C₁-C₆ saturated aliphatic hydrocarbons such as methane, ethane, propane and butene, mixtures thereof, or other oxidizing gases.

In certain exemplary embodiments of the present invention, the atmosphere during thermal treatment is substantially free of oxygen. The oxygen content may be preferably below about 10 ppm, or more preferably below about 1 ppm.

The composite material obtained by thermal treatment can be further treated with suitable oxidizing and/or reducing agents, including treatment of the material at elevated temperatures in oxidizing atmospheres. Examples of oxidizing atmospheres include air, oxygen, carbon monoxide, carbon dioxide, nitrogen oxides, or similar oxidizing agents. The gaseous oxidizing agent can also be mixed with inert gases such as nitrogen, or noble gases such as argon. Partial oxidation of the resultant composite materials can be accomplished at elevated temperatures in the range of about 50 C to 800 C, in order to modify the porosity, pore sizes and/or surface properties. Besides partial oxidation of the material with gasous oxidizing agents, liquid oxidizing agents can also be applied. Liquid oxidizing agents can include, for example, concentrated nitric acid. Concentrated nitric acid can contact the composite material at temperatures above room temperature. Suitable reducing agents such as hydrogen gas or the like, may be used to reduce metal compounds to the zero-valent metal after the conversion step.

In further exemplary embodiments of the present invention, high pressure may be applied to form the composite material. The conversion step may be performed by drying under supercritical conditions, for example in supercritical carbon dioxide, which can lead to highly porous aerogel composites. Reduced pressure or a vacuum may also be applied to convert the sol/gel into the composite material.

Suitable conditions, such as temperature, atmosphere, and/or pressure, may be applied depending on the desired property of the final composite material and the components used to form the material. The polymer-encapsulated metal-based compounds may still be present in the formed composite material without having decomposed, depending on the conversion conditions used.

By oxidative and/or reductive treatment or by the incorporation of additives, fillers or functional materials, the properties of the composite materials produced can be influenced and/or modified in a controlled manner. For example, it is possible to render the surface properties of the composite material hydrophilic or hydrophobic by incorporating inorganic nanoparticles or nanocomposites such as layer silicates.

According to further exemplary embodiments of the present invention, it is possible to suitably modify the composite material, e.g. by varying the pore sizes using suitable oxidative or reductive after-treatment steps, including but not limited to oxidation in air at elevated temperatures, boiling in oxidizing acids or alkalis, or admixing volatile components which can be degraded completely during the conversion step, thereby possibly leaving pores behind in the carbon-containing layer.

Coatings or bulk materials may be structured in a suitable way before or after conversion into the composite material by folding, embossing, punching, pressing, extruding, gathering, injection molding, and the like, either before or after being applied to the substrate or being molded or formed. In this way, certain structures of a regular or irregular type can be incorporated into coatings produced with the composite material.

The combination materials can be further processed by conventional techniques, e.g., they can be used to build molded paddings and the like, or to form coatings on any substrates including but not limited to implants such as stents, bone substitutes, and the like.

Molded paddings can be produced in almost any desired form. The molded paddings may be in the form of pipes, bead-moldings, plates, blocks, cuboids, cubes, spheres or hollow spheres, or any other three-dimensional structure which may be, for example, longish, circle-shaped, polyether-shaped, e.g. triangular, bar-shaped, plate-shaped, tetrahedral, pyramidal, octahedral, dodecahedral, icosahedral, rhomboidal, prismtic, or in round shapes such as ball-shaped, spheroidal or cylindrical, lens-shaped, ring-shaped, honeycomb-shaped, and the like.

By applying multi-layered half-finished molded shapes, asymmetric constructions can be formed from the composite materials. The materials can be brought into the desired form by applying any appropriate conventional technique, including but not limited to casting processes such as sand casting, shell molding, full mold processes, die casting, centrifugal casting, or by pressing, sintering, injection molding, compression molding, blow molding, extrusion, calendaring, fusion welding, pressure welding, jiggering, slip casting, dry pressing, drying, firing, filament winding, pultrusion, lamination, autoclave, curing or braiding.

Coatings formed from sols/combinations may be applied in liquid, pulpy or pasty form, for example, by painting, furnishing, phase-inversion, dispersing atomizing or melt coating, extruding, slip casting, dipping, or as a hot melt. Where the combination is in a solid state, it may be applied as a coating onto a suitable substrate using such techniques as, e.g., powder coating, flame spraying, sintering, or the like. Dipping, spraying, spin coating, ink-jet-printing, tampon and microdrop coating or 3-D-printing may also be used. The coating may be applied to an inert substrate, dried and, if necessary, thermally treated, where the substrate may be thermally stable, or it may be thermally instable yielding a substantially complete degradation of the substrate, such that only the coating remains in the form of the composite material after thermal treatment.

Combination sols or gels can be processed by any appropriate conventional technique. Preferred techniques may include folding, stamping, punching, printing, extruding, die casting, injection molding, reaping, and the like. Coatings may also be formed by a transfer process, in which the combination gels are applied to the substrates as a lamination. The coated substrates can be cured, and subsequently the coating can be released from the substrate to be thermally treated. The coating of the substrate can be provided by using suitable printing procedures, e.g. gravure printing, scraping or blade printing, spraying techniques, thermal laminations, or wet-in-wet laminations. It is possible to successively apply a plurality of thin layers to a substrate to provide a more uniform and thicker coating of composite film.

By applying the above-mentioned transfer procedure, it is also possible to form multi-layer gradient films by using different material layers and different sequences of layers. Conversion of these multilayer coatings into a composite material can provide gradient materials, wherein the density and other properties may vary from place to place.

In another exemplary embodiment of the present invention, the combination according to the invention may be dried or thermally treated and commuted by suitable conventional techniques, for example by grinding in a ball mill or roller mill and the like. The commuted material can be used as a powder, a flat blank, a rod, a sphere, a hollow sphere in different grainings, and the like, and can be further processed by conventional techniques known in the art to form granulates or extrudates in various forms. Hot-pressure-procedures, accompanied by suitable binders as appropriate, can also be used to form the composite materials.

Additional processing options can include, but are not limited to, the formation of powders by other conventional techniques such as spray-pyrolysis, precipitation, and formation of fibers by spinning-techniques such as gel-spinning.

The structures of the composite materials, in particular ceramic and composite half-finished materials, molded paddings and coatings, as well as substantially pure metal-based materials, e.g. mixed metal oxides, can range from amorphous to fully crystalline depending on the temperature and the atmosphere chosen for the thermal treatment, and on the specific composition of the components used to produce the composite materials.

The porosity and the pore sizes may also be varied over a wide range, simply by varying the components in the sol and/or by varying the particle size of the encapsulated metal-based compounds.

Furthermore, by suitable selection of components and processing conditions, bioerodible coatings or coatings and materials which are dissolvable or may be peeled off from substrates in the presence of physiologic fluids can be produced. For example, coatings comprising composite materials may be used for coronary implants such as stents, wherein the coating further comprises an encapsulated marker, e.g., a metal compound having signaling properties and thus may produce signals detectable by physical, chemical or biological detection methods such as x-ray, nuclear magnetic resonance (NMR), computer tomography methods, scintigraphy, single-photon-emission computed tomography (SPECT), ultrasonic, radiofrequency (RF), and the like. Metal compounds used as markers may be encapsulated in a polymer shell or coated thereon and thus can be prevented from interfering with the implant material, which can also be a metal, where such interrference can often lead to electrocorrosion or related problems. Coated implants may be produced with encapsulated markers, wherein the coating remains permanently on the implant. In one exemplary embodiment of the present invention, the coating may be rapidly dissolved or peeled off from a stent after implantation under physiologic conditions, allowing a transient marking to occur. An exemplary embodiment is described in example 7 below, wherein encapsulated metal-based compounds such as those discussed herein, e.g., dextrane coated iron particles, are incorporated into a silica sol of any of the materials discussed above, converted into an aerogel, which may be in particle form or applied to an implant as a coating, wherein the aerogel may be dissolvable in body fluids and thereby release the iron particles. This coating may additionally incorporate drugs, such as paclitaxel in example 7, and thus may permit monitoring of the drug concomittantly released with the metal marker from an implant or a coating of an implant, by non-invasive detection methods, further allowing for the determination of the extent and regional distribution of the drug released.

If therapeutically active compounds are used in forming the composite materials, they may preferably be encapsulated in bioerodible or resorbable polymers, allowing for a controlled release of the active ingredient under physiological conditions.

The invention will now be further described by way of the following non-limiting examples. Analyses and parameter determination in these examples were performed by the following methods:

Particle sizes are provided as mean particle sizes, as determined on a CIS Particle Analyzer (Ankersmid) by the TOT-method (Time-Of-Transition), X-ray powder diffraction, or TEM (Transmission-Electron-Microscopy). Average particle sizes in suspensions, emulsions or dispersions were determined by dynamic light scattering methods. Average pore sizes of the materials were determined by SEM (Scanning Electron Microscopy). Porosity and specific surface areas were determined by N₂ or He absorption techniques, according to the BET method.

### EXAMPLE 1

In a mini-emulsion polymerization reaction, 5.8 g of deionized water, 5.1 mM of acrylic acid (obtained from Sigma Aldrich), 0.125 mol of methylmethacrylic acid MMA (obtained from Sigma Aldrich) and 9.5 g of a 15 weight % aqueous solution of SDS surfactant (obtained from Fischer Chemical) were introduced into a 250 ml four-neck-flask equipped with a reflux condenser under a nitrogen atmosphere (nitrogen flow 2 liters/min.). The reaction mixture was stirred at 120 rpm for 1 hour while heated in an oil bath at 85° C. until a stable emulsion had formed. 0.1 g of an ethanolic iridium oxide sol (concentration 1 g/l) having an average particle size of 80 nm were added to the emulsion and the mixture was stirred for another 2 hours. Then, a starter solution comprising 200 mg of potassium peroxodisulfate in 4 ml of water was slowly added over a time period of 30 minutes. After 4 hours, the mixture was neutralized to pH 7 and the resulting mini-emulsion of encapsulated iridium oxide particles was cooled to room temperature. The average particle size of the encapsulated iridium oxide particles in the emulsion were about 120 nm. The emulsion was dried in vacuo for 72 hours, and a suspension of the resulting encapsulated particles in ethanol having a concentration of 5 mg/ml was prepared.

A homogeneous sol was prepared from 100 ml of a 20 weight % solution of magnesium acetate tetrahydrate (Mg(CH₃COO)₂×4H₂O in ethanol and 10 ml of a 10% nitric acid at room temperature by stirring for 3 hours. 4 ml of tetraethoxy-orthosilane TEOS (obtained from Degussa) were added to the sol and the mixture was stirred at a room temperature for another 2 hours at 20 rpm. 2 ml of the sol and 2 ml of the ethanol suspension of the encapsulated iridium oxide particles as prepared above were combined and stirred at room temperature for 30 hours at 20 rpm. Subsequently, the combination was sprayed as a thin layer onto three substrates: a metallic substrate, a ceramic substrate, and a glass substrate, each in the form of a 2 cm×2 cm sample. The coated substrates were transferred into a tube furnace and thermally treated in an air atmosphere at 350° C. for a period of 4 hours. After cooling to room temperature, the three exemplary samples each exhibited a rough-textured, tightly adhering, hazy coating. Analysis by scanning electronic microscopy SEM revealed that the coating was porous, having an average pore size of about 80 nm.

### EXAMPLE 2

A mini-emulsion was prepared as in example 1 above. However, the amount of surfactant used was reduced to 0.25 g of the 15% aqueous SDS solution in order to enlarge the resulting PMMA capsules. The resulting PMMA-encapsulated iridium oxide particles had a mean particle size of 400 nm. The emulsion was dried in vacuo for 72 hours, and a suspension of the encapsulated particles in ethanol having a concentration of 5 mg/ml was prepared.

In accordance with the procedure outlined in example 1 above, a homogeneous sol was produced from 100 ml of a 20 weight % solution of magnesium acetate tetrahydrate in ethanol, followed by the addition of 10 ml of a 10% nitric acid at room temperature and stirring for 3 hours, then adding 4 ml of TEOS (obtained from Degussa) and stirring at 20 rpm for an additional 2 hours at room temperature. 2 ml of the sol and 2 ml of the suspension of encapsulated iridium oxide were combined, stirred for 30 minutes at room temperature at 20 rpm, and subsequently sprayed onto a metallic substrate, a ceramic substrate, and a glass substrate as in example 1 above. The coated substrates were then transferred into a tube furnace and thermally treated in an air atmosphere at 350° C. for a period of 4 hours. The resulting samples were cooled to room temperature and each substrate exhibited a rough-textured, tightly adhering, hazy coating. Analysis by SEM revealed a porous coating having an average pore size of about 250 nm.

### EXAMPLE 3

In a mini-elmulsion polymerisation reaction, 5.8 g of deionized water, 5.1 mM of acrylic acid (obtained from Sigma Aldrich), 0.125 mol of metylmethacrylic acid (also obtained from Sigma Aldrich) and 0.5 g of a 15 weight % aqueous solution of SDS surfactant (obtained from Fischer Chemical) were combined in a 250 ml four-neck-flask equipped with a flask condenser under a nitrogen atmosphere (providing a nitrogen flow of 2 liters/min.) and stirred at 120 rpm for about 1 hour in an oil bath at 85° C., to obtain a stable emulsion. To the emulsion, 0.1 g of an ethanolic magnesium oxide sol (concentration 2 g/l) having a mean particle size of 15 nm was added and the mixture was stirred for another 2 hours. Subsequently a starter solution comprising 200 mg potassium peroxodisulfate in 4 ml of water was slowly added over 30 minutes. After 4 hours the mixture was neutralized to pH 7 and the resulting mini-emulsion of PMMA-encapsulated magnesium oxide particles was cooled to room temperature. The resulting emulsion had a mean particle size of about 100 nm. The emulsion was dried in vacuo for 72 hours, providing PMMA-encapsulated MgO particles.

A homogenous sol was then prepared from 100 ml of a 20 weight % solution of magnesium acetate tetrahydrate in ethanol to which 10 ml of 10% nitric acid was added at room temperature, and the mixture was stirred for 3 hours. To the sol 1 ml of Tween® 20 was added as a surfactant, and 1.5 mg of magnesium oxide powder and 15 mg of the PMMA-encapsulated magnesium oxide particles as prepared above were added with continuous stirring. To accelerate gelation, 2 mg of glycerine were added and the viscous mixture was poured into a metallic mold. After drying in a convection oven, the molded padding was treated in a thermolysis process at 350° C. in an air atmosphere for 8 hours in a tube furnace. The resulting molded paddings consisting primarily of magnesium oxide revealed a porosity of 60% with a mean pore size of 60 nm.

### EXAMPLE 4

A mini-emulsion was produced according to the process described in example 3 above, with the amount of surfactant reduced to 0.25 g of the 15% SDS solution in order to increase the size of the PMMA capsules. The resulting PMMA-encapsulated magnesium oxide particles had a mean particle size of about 350 nm. The emulsion was dried for 72 hours in vacuo, resulting in dried capsules containing MgO.

A homogeneous sol was then produced from 100 ml of a 20 weight % solution of magnesium acetate tetrahydrat ethanol, subsequently adding 10 ml of 10% nitric acid at room temperature and stirring for a period of 3 hours. Next, 1 ml of Tween® 20 as a surfactant, 1,5 mg of magnesium oxide powder, and 15 mg of the encapsulated magnesium oxide particles as prepared above were added while stirring. For accelerating the gelation, 2 mg of glycerine were added and the viscous mixture was poured into a metallic mold. After drying in a convection oven, the molded padding was treated in a thermolysis process at 350° C. in an air atmosphere for 8 hours in a tube furnace. The resulting molded padding consisted primarily of magnesium oxide, and exhibited a porosity of 50% and a mean pore size of 180 nm.

### EXAMPLE 5

In a mini-emulsion polymerisation reaction, 5.8 g of deionized water, 5.1 mM of acrylic acid (obtained from Sigma Aldrich), 0.125 mol of methylmethracrylic acid MMA (also obtained from Sigma Aldrich) and 0.5 g of a 15 weight % aqueous solution of SDS surfactant (obtained from Fischer Chemical) were introduced into a 250 ml four-neck-column equipped with a reflux condenser under a nitrogen atmosphere (using a nitrogen flow of 2 liters/min.). The mixture was stirred at 120 rpm for 1 hour in an oil bath at 85° C. to obtain a stable emulsion. To the emulsion was added 0.05 g of an ethanolic magnesium oxide sol with a mean particle size of 15 nm, 0.05 g of an ethanolic dispersion of iridium oxide nano particles having a mean particle size of 60 nm, 0.05 g of an ethanolic dispersion of tantalum carbide particles having a mean particle size of 160 nm, and 0.05 g of an ethanolic zirconium dioxide dispersion having a mean particle size of 25 nm (each dispersion having a concentration of 2 g/l), and the resulting mixture was stirred for an additional 2 hours. Then, a starter solution consisting of 200 mg of potassium peroxodisulfate in 4 ml water was slowly added over 30 minutes. After 4 hours the mixture was neutralized to pH 7 and the resulting mini-emulsion with the encapsulated mixed oxide particles was cooled to room temperature. The capsules in the emulsion had a mean particle size of 200 nm. The emulsion was dried for 72 hours in vacuo, and an ethanol suspension of the dried particles having a concentration of 5 mg/l was produced.

A homogenous sol was then prepared from 300 g of tetramethylorthosilan TMOS (obtained from Degussa) and 300 g of deionized water, 3 g of Tween® 20 as a surfactant, and 1 g of 1-N-HCl as a catalyst, which was stirring for 30 minutes at room temperature. 5 ml of this sol were combined with 5 ml of the ethanolic suspension of the encapsulated mixed oxide particles, and the resulting mixture was stirred for 6 hours and subsequently sprayed onto metallic, ceramic, and quartz glass substrates as described above. Thereafter, the samples were sinterd at 700° C. for 4 hours. The resulting mixed-metal oxide composite coating exhibited a porosity of 40% and a mean particle size of 50 nm.

### EXAMPLE 6

An ethanol suspension of PMMA encapsulated iridium oxide particles was prepared at a concentration of 5 mg/ml, as described in example 1 above.

A sol was then prepared, also as described in example 1. 2 ml of the sol was combined with 2 ml of the ethanolic suspension of encapsulated iridium oxide, stirred for 30 minutes at room temperature (20 rpm), and subsequently sprayed onto commercially available metallic stents (KAON 18.5 mm, Fortimedix) and dried at 120° C. A solid elastic coating was obtained. The coated stents were introduced into a beaker and agitated in a PBS buffer solution at 37.5° C. at 75 rpm. Within 5 hours, the coating peeled off from the stents and the PMMA encapsulated iridium oxide particles were found in the sediment formed at the bottom of the beaker. This confirmed the suitability of such encapsulated iridium oxide coatings as transient marker substances which may be rapidly dissolved or peeled of from the stent, for example, after insertion into the human body.

### EXAMPLE 7

300 g of tetramethylorthosilane (obtained from Degussa) were stirred together with 300 g of deionized water and 1 g of 1N HCl as a catalyst for 30 minutes at room temperature in a glass vessel, so that a homogeneous sol was produced. 3 ml of the sol was combined with 3 ml of a suspension containing dextran-coated paramagnetic iron oxide particles having particle sizes of 80-120 nm (as specified by the manufacturer) of a commercial MRI contrast agent (Endorem, obtained from Laboratoire Guerbet), wherein the concentration of the paramagnetic iron (II-, III-) oxide particles was set at 5 mg/ml through dilution in physiological salt solution, and gelled at room temperature for a period of 5 days in 2 ml Eppendorf cups and dried under vacuum. The lightly dulled aerogels thus prepared, having spherical form with radiolucent and paramagnetic, biodegradable properties, and having a volume of about 0.8 ml, were incubated by shaking (at 75 rpm) for 30 days at 37.5° C. in 4 ml of PBS buffer solution, wherein the buffer supernatant was removed daily and replaced with fresh buffer solution. The amount of iron released from the supernatant was determined by means of flame atomic absorption spectrometry. The average release rate of the iron particles released in the implant body amounted to 6-8% of the total amount per day, and correlated with the dissolution of the aerogel bodies in the buffer solution.

In a further test, 300 g of tetramethylorthosilane (obtained from Degussa) were stirred together with 300 g of deionized water and 1 g of 1N HCl as a catalyst for 30 minutes at room temperature in a glass vessel, so that a homogeneous sol was produced. 5 ml of the sol were combined with 1.5 ml of a suspension containing dextran-coated paramagnetic iron oxide particles having particle sizes of 80-120 nm (per manufacturer) of a commercial MRI contrast agent (Endorem, obtained from Laboratoire Guerbet), wherein the concentration of the paramagnetic iron (II-, III-) oxide particles was set at 5 mg/ml by means of dilution in physiological salt solution, and additionally combined with 2.5 ml of a 6% ethanolic Paclitaxel solution and gelled at room temperature for a period of 5 days in 2 ml Eppendorf cups and dried under vacuum. The lightly dulled aerogels thus prepared, having spherical form with radiolucent, paramagnetic, biodegradable and active substance releasing properties, and having a volume of about 1.2 ml, were incubated while shaking (75 rpm) for 30 days at 37.5° C. in 4 ml of PBS buffer solution, wherein the buffer supernatant was removed daily and replaced with fresh buffer solution. The amount of iron released from the supernatant was determined by flame atomic absorption spectrometry and the amount of Paclitaxel released was determined by HPLC. The average release rate of the iron particles into the implant body amounted to 6-8% of the total amount per day, and correlated with the average released amount of Paclitaxel released per day of 5-10%, and also correlated with the dissolving of the aerogel body into the buffer solution.

Having thus described in detail several exemplary embodiments of the present invention, it is to be understood that the invention described above is not to be limited to particular details set forth in the above description, as many apparent variations thereof are possible without departing from the spirit or scope of the present invention. The embodiments of the present invention are disclosed herein or are obvious from and encompassed by the detailed description. The detailed description, given by way of example, is not intended to limit the invention solely to the specific embodiments described.

The foregoing applications and all documents cited therein or during their prosecution (“appln. cited documents”) and all documents cited or referenced in the appln. cited documents, and all documents, references and publications cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the invention. Citation or identification of any document in this application is not an admission that such document is available as prior art to the present invention. It is noted that in this disclosure and particularly in the claims, terms such as “comprises,” “comprised,” “comprising” and the like can have the meaning attributed to them in U.S. Patent law; e.g., they can mean “includes,” “included,” “including” and the like; and that terms such as “consisting essentially of” and “consists essentially of” can have the meaning ascribed to them in U.S. Patent law, e.g., they allow for elements not explicitly recited, but exclude elements that are found in the prior art or that affect a basic or novel characteristic of the invention.

What is claimed is:

1. A process for manufacturing a metal-containing composite material, comprising:
a) providing at least one first composition comprising at least one metal-based compound and at least one polymer;
b) forming a sol from sol/gel forming components;
c) combining the at least one first composition with the sol to produce a second composition; and
d) converting the second composition into the metal-containing composite material.

2. The process of claim 1, wherein the at least one first composition is a polymer-encapsulated metal-based compound.

3. The process of claim 2, further comprising encapsulating at least one metal-based compound in a polymeric shell to form the at least one first composition.

4. The process of claim 2, wherein the at least one metal-based compound is in a form of a colloidal particle.

5. The process of claim 1, wherein the at least one first composition is a polymeric particle coated with the at least one metal-based compound.

6. The process of claim 1, wherein step (b) is performed using a hydrolytic sol/gel-process in the presence of water.

7. The process of claim 1, wherein step (b) is performed using a non-hydrolytic sol/gel-process in the absence of water.

8. The process of claim 1, wherein the at least one metal-based compound includes at least one of zero-valent metals, metal alloys, metal oxides, inorganic metal salts, organic metal salts, organometallic compounds, metal alkoxides, semiconductive metal compounds, metal carbides, metal nitrides, metal oxynitrides, metal carbonitrides, metal oxycarbides, metal oxynitrides, metal oxycarbonitrides, metal-based core-shell nanoparticles, metal-containing endohedral fullerenes, or endometallofullerenes.

9. The process of claim 8, wherein the at least one metal-based compound is in a form of at least one of a nanocrystalline particle, a microcrystalline particle, or a nanowire.

10. The process of claim 9, wherein the at least one metal-based compound has an average particle size that is between about 0.5 nm and 1000 nm.

11. The process of claim 9, wherein the at least one metal-based compound has an average particle size that is between about 0.5 nm and 900 nm.

12. The process of claim 9, wherein the at least one metal-based compound has an average particle size that is between about 0.7 nm and 800 nm.

13. The process of claim 1, wherein the sol/gel forming components include at least one of alkoxides, metal alkoxides, metal oxides, metal acetates, metal nitrates, or metal halides.

14. The process of claim 13, wherein the sol/gel forming components include at least one of silicon alkoxides, tetraalkoxysilanes, oligomeric forms of tetraalkoxysilanes, alkylalkoxysilanes, aryltrialkoxysilanes, (meth)acrylsilanes, phenylsilanes, oligomeric silanes, polymeric silanes, epoxysilanes; fluoroalkylsilanes, fluoroalkyltrimethoxysilanes, or fluoroalkyltriethoxysilanes.

15. The process of claim 1, wherein step (b) is performed in the presence of an organic solvent, and the sol comprises between about 0.1% and 90% organic solvent.

16. The process of claim 1, wherein step (b) is performed in the presence of an organic solvent, and the sol comprises between about 1% and 90% organic solvent.

17. The process of claim 1, wherein step (b) is performed in the presence of an organic solvent, and the sol comprises between about 5% and 90% organic solvent.

18. The process of claim 1, wherein wherein step (b) is performed in the presence of an organic solvent, and the sol comprises between about 20% and 70% organic solvent.

19. The process of claim 3, wherein the metal-based compound is encapsulated in a polymer material which includes at least one of poly(meth)acrylate, polymethylmethacrylate, unsaturated polyester, saturated polyester, polyolefines, polyethylene, polypropylene, polybutylene, alkyd resins, epoxy-polymers, epoxy resins, polyamide, polyimide, polyetherimide, polyamideimide, polyesterimide, polyesteramideimide, polyurethane, polycarbonate, polystyrene, polyphenole, polyvinylester, polysilicone, polyacetale, cellulosic acetate, polyvinylchloride, polyvinylacetate, polyvinylalcohol, polysulfone, polyphenylsulfone, polyethersulfone, polyketone, polyetherketone, polybenzimidazole, polybenzoxazole, polybenzthiazole, polyfluorocarbons, polyphenylenether, polyarylate, or cyanatoester-polymere.

20. The process of claim 3, wherein the metal-based compound is encapsulated in an elastomeric polymer material which includes at least one of polybutadiene, polyisobutylene, polyisoprene, poly(styrene-butadiene-styrene), polyurethane, polychloroprene, silicone, or copolymers of any of the foregoing.

21. The process of claim 2, wherein the metal-based compound is encapsulated in at least one of a plurality of shells or layers of organic material.

22. The process of claim 3, wherein the at least one metal-based compound is further encapsulated in at least one of a vesicle, a liposome, a micelle, or an overcoat of a suitable coating material.

23. The process of claim 2, further comprising chemically modifying the at least one first composition by at least one of a suitable linker group or a coating, which is capable of reacting with the sol/gel forming components.

24. The process of claim 1, wherein the at least one metal-based compound and at least one of the sol/gel forming components are substantially the same.

25. The process of claim 1, wherein at least one of the sol/gel forming components is a metal-based compound encapsulated in a polymeric shell.

26. The process of claim 2, further comprising adding at least one further additive to at least one of the at least one first composition, the sol, or the second composition.

27. The process of claim 26, wherein the at least one further additive includes at least one of biologically active compounds, therapeutically active compounds, fillers, surfactants, acids, bases, crosslinkers, pore-forming agents, plasticizers, lubricants, flame resistant materials, glass, glass fibers, carbon fibers, cotton, fabrics, metal powders, metal compounds, silicon, silicon oxides, zeolites, titanium oxides, zirconium oxides, aluminum oxides, aluminum silicates, talcum, graphite, soot, phyllosilicates, drying-control chemical additives, glycerol, DMF, or DMSO.

28. The process of claim 1, wherein step (d) comprises drying the second composition.

29. The process of claim 28, wherein the second composition is dried using a thermal treatment in a range of about −200° C. to 3500° C.

30. The process of claim 29, wherein the thermal treatment is performed under at least one of a reduced pressure or a vacuum.

31. The process of claim 1, wherein step (d) comprises at least one of performing a pyrolysis or a sintering heat treatment of the second composition at temperatures up to about 3500° C.

32. The process of claim 2, further comprising adding at least one crosslinking agent to at least one of the at least one first composition, the sol, or the second composition, wherein the crosslinking agent includes at least one of isocyanates, silanes, (meth)acrylates, 2-hydroxyethyl methacrylate, propyltrimethoxysilane, 3-(trimethylsilyl)propyl methacrylate, isophoron diisocyanate, HMDI, diethylenetriaminoisocyanate, 1,6-diisocyanatohexane, or glycerine.

33. The process of claim 2, further comprising adding at least one filler to at least one of the at least one first composition, the sol, or the second composition, wherein the at least one filler is incapable of reacting with the sol/gel forming components.

34. The process of claim 33, wherein the at least one filler is a non-polymeric material that includes at least one of inorganic salts, cationic surfactants, anionic surfactants, or non-ionic surfactants,

35. The process of claim 33, wherein the at least one filler includes at least one of polymer-encapsulated carbon species, polymer-encapsulated fullerenes, polymer-encapsulated nanotubes, polymer-encapsulated onions, metal-containing soot, graphite, diamond particles, carbon black, or carbon fibers.

36. The process of claim 33, further comprising at least partially removing the filler from the solid metal-containing composite material.

37. The process of claim 36, wherein the at least partially removing the filler comprises at least one of dissolving the filler in at least one of water, diluted mineral acids, concentrated mineral acids, diluted mineral bases, concentrated mineral bases, diluted organic acids, concentrated organic acids, diluted organic bases, concentrated organic bases, or organic solvents, or thermally decomposing the filler at least one of during or after converting the second composition.

37. A metal-containing composite material produced by the steps comprising:
a) providing at least one first composition comprising a metal-based compound and a polymer;
b) forming a sol from sol/gel forming components;
c) combining at least one first composition with the sol to produce a second composition; and
d) converting the second composition into a metal-containing composite material.

38. The metal-containing composite material of claim 37, wherein the material is in the form of a coating. or as a bulk material.

39. The metal-containing composite material of claim 37, wherein the material is in the form of a bulk material.

40. The metal-containing composite material of claim 37, wherein the material has bioerodible properties in the presence of physiologic fluids.

41. The metal-containing composite material of claim 37, wherein the material is at least partially dissolvable in the presence of physiologic fluids.
